(19) United States
(12) Patent Application Publication
(10) Pub. No.: US 2016/0157703 A1
(43) Pub. Date: Jun. 9, 2016

(54) TISSUE GRIPPING DEVICE

(71) Applicant: THE HOSPITAL FOR SICK CHILDREN, Toronto (CA)

(72) Inventors: Robert Brooks, Sarnia (CA); Thomas Looi, Markham (CA); James Drake, Toronto (CA)

(21) Appl. No.: 14/900,049

(22) PCT Filed: Jun. 18, 2014
(86) PCT No.: PCT/CA2014/050573

§ 371 Date: Dec. 18, 2015

Related U.S. Application Data
(60) Provisional application No. 61/836,478, filed on Jun. 18, 2013.

(51) Int. Cl. A61B 1/00; A61B 17/128

(52) U.S. Cl. CPC A61B 1/00094; A61B 17/1285; A61B 1/00137; A61B 1/0014; A61B 1/00087

(57) ABSTRACT

The present disclosure provides a tissue gripping device. The device comprises a head with contact face attachable to the distal end of at least one hollow member. The contact face has one or more openings in flow communication with a suction device though the at least one hollow member wherein a negative pressure gradient can be applied across the one or more openings to allow the head to grasp tissue. The device may comprise flexible members to facilitate the navigation of internal organs. Additionally, the head can have one or more tool holes. The tool hole can house an ejecting surgical clip for medical procedure. The device may further be attachable to a sensor or another medical device. The grasping allows fixation and manipulation of tissue, traversal of the small intestine, and counter traction for tool insertion.

### FIELD OF THE DISCLOSURE

The present disclosure relates to a device to accomplish the surgical task of grasping tissue inside hollow viscera and other hollow organs. Additionally, the grasping of tissue provides the counter traction for operations such as clip application, biopsy, and the like. Finally, the grasping of tissue can be used to provide traversal of the small intestine.

### BACKGROUND

Natural orifice surgery is a common method of treating diseases of the hollow organs where we have a natural opening into the body. Currently, endoscopes are inserted into these natural orifices and used to navigate to the site of interest. They commonly have a single tool hole with which to insert a wide range of tools such as, but not limited to, electrocautery, clips, or biopsy needles. However, the current state of endoscopes doesn't offer a way to fix the tissue in place while performing these actions.

Additionally, without countertraction, it is extremely difficult to pass a suture through an organ wall and thus all endoscopic clips clamp the tissue rather than passing through the tissue in a loop like a regular suture; this leaves the clipped tissue protruding from the organ wall and can act as an obstruction to flow through the organ.

Finally, access to the small intestine is difficult and requires a device to pull the small intestine over the endoscope; this is known as enteroscopy. Currently, balloon enteroscopy is used where a balloon is inflated around the scope to fix the small intestine, the scope is advanced, the balloon is deflated and advanced, the balloon is re-inflated, both the endoscope and balloon are pulled back, and then the process repeats thereby advancing the scope down the small intestine. Unfortunately, this is a very slow process and the small intestine is long, resulting in long surgeries.

A system was disclosed in U.S. Pat. No. 8,206,295, herein referred to as the suction pad that uses suction applied to holes on the side of the tool to grasp and manipulate tissue in the digestive tract. However this system is different from embodiments of the present disclosure in many respects. The suction pad was designed to enter the body through a laparoscopic port where as our system can enter through both a laparoscopic port and a natural orifice such as, but not limited to, the mouth. The suction pad does not attach or integrate into an existing endoscope which necessitates additional or larger entries into the body. The suction pad must be rolled up to enter the body and does not disclose a method of removal from the body using the same port size where as our system enters and exits from a natural orifice or port by itself or attached or integrated into an endoscope with or without collapsing. The suction pad cannot translate, rotate, or pitch axially without the aid or external graspers which requires additional entries into the body and additional tools. The suction pad grasping surface is flexible and deformable where as ours is rigid when in use; although, the surface may be soft or the grasping head may be expandable. A fixed shape face allows faster grasping since the tool does not need to be deformed to the tissue surface. The suction pad does not operate in small tubular organs such as, but not limited to, the esophagus or small intestine. The suction pad is a singular entity with two and only two suction chambers whereas our system can act with many head each with one or more suction chambers where each chamber can be individually or synchronously pressurized and each head can be translated, rotated, or pitch which respect to one another or an endoscope. Our suction system is designed to provide counter traction for a tool hole by surrounding set tool hole with suction holes. This allows, but is not limited to, placement of surgical clips or precision biopsy or injection. Our suction system facilitates viewing through the device and many different sensors and imaging types. Our system can use the suction to form a seal around a tissue for local application of collapsed mesh structures, fluid, gel, gas, plasma, drugs, growth factors, cells or any combination thereof. The suction pad only rolls up and still occupies the same volume whereas our system can collapse and occupies less volume.

Additionally, a patent U.S. Pat. No. 7,507,235 discloses a suction tissue stabilization device for the heart which also differs from our device as follows. A tool channel, surgical clip, or surgical clip applier are not disclosed; only a cannula for drug delivery and energy delivery electrodes. They specify a rigid face that is specially shaped to the target tissue but they don't talk about anatomy-specific, age-specific, or patient-specific and never discuss using pre-operative imaging to select sizes. A mixture of hole sizes or a special arrangement of holes is never disclosed; although they try to claim “any suitable arrangement” but our arrangements are for special purposes which are non-obvious. Use of an expandable bladder for either grasping or expansion/contraction is not disclosed. They refer to microgrooves but don't specify soft microgroove which are required to achieve the instance where the pillars flatten to increase surface area which is non-obvious. Finally, the support structure is not integrated with the functional channels (suction, irrigation, etc.) and the functional channels are not used for actuation.

### SUMMARY

Disclosed herein is a tissue gripping device for use in association with a clip having a distal end and a suction device, comprising a hollow member having a distal end and a proximal end; a head, having a contact face and an inlet, the inlet being attachable to the distal end of the hollow member; a tool channel having a distal end and a proximal end; the contact face having at least one opening, the at least one opening being in flow communication with a suction device through the hollow member; a generally elongate tool housing formed in the head and configured to receive the clip wherein the tool housing is attachable to and in flow communication with the distal end of the tool channel; a tool trench formed in the head configured to receive therein the distal end of the clip; and a control device attachable to the proximal end of the hollow member and operably attachable to the proximal end of the tool channel, whereby the clip is ejected responsive to actuation by the control device from the tool channel and into the tool trench.

In addition, the clip may be loaded into the tool housing through the proximal end of the tool channel. The clip may have a loop shape in stable equilibrium position. The clip may be composed of elastic, super-elastic, or shape memory material. The clip may be composed of self-closing, super-elastic nitinol. The tool housing may have an embedded ejection mechanism which may be actuated hydraulically, electro-dynamically or pneumatically from the control device.

Another embodiment disclosed herein is a tissue gripping device for use in association with a suction device and a bladder pump, comprising: a hollow member having a distal end and a proximal end; a head having a contact face and an inlet, wherein the inlet is attachable to the distal end of the hollow member; an inflatable bladder, formed in the head, in flow communication with the bladder pump; the contact face having at least one opening wherein the at least one opening is in flow communication with the suction device through the hollow member; and a control device attachable to the proximal end of the hollow member.

In addition, the dimensions of the head may be increased or decreased responsive to the bladder pump. Furthermore, the dimensions of the openings may be increased or decreased by a similar mechanism. The inflatable bladder may be inflated or deflated hydraulically or pneumatically.

Another embodiment disclosed herein is a tissue gripping device for use in association with a suction device, comprising: at least two members each having a distal end and a proximal end, wherein one of the at least two members is a flexible member and wherein one of the at least two members is a hollow member; a head, having a contact face and an inlet, where the inlet is attachable to the distal end of the hollow member; the contact face having at least one opening wherein the at least one opening is in flow communication with the suction device through the hollow member; and a control device attachable to the proximal end of the at least two members.

In addition, the hollow members described above may be flexible such that translation or rotation of the members may achieve translation or rotation of the head. The hollow members may be reinforced with a spring core or a super-elastic tube. The head may further comprise one or more attachable chords, ropes, strings or wires such that applied tension may translate or rotate the head. The contact face may have a convex shape.

Furthermore the contact shape may be chosen to match the radius of concavity of an organ, an orifice or a lumen. The hollow member may act as a conduit for flow communication between the openings in the face and the suction device. The suction device may be a pump or a vacuum. The surface of the contact face may be treated with microgrooves. The microgrooves may be stiff micro-pillars or soft micro-pillars that deform under sheering or normal force. The surface may be treated with a hydrophilic, hydrophobic, knurled or abraded coating. The head may be rigid. A lens, window or fiber optic cable may be built into the head or the head may be constructed out of transparent or translucent material to aid in viewing the operation site. Different sized openings may be used such that smaller openings are located closer to the perimeter of the contact face. The device may be attachable a tool selected from the list including but not limited to: an endoscope, a drug administering device, an electrode or a medical retractor. The device may be attachable to a sensor selected from the list including but not limited to: a force sensor, a pressure sensor, a capacitance sensor, a temperature sensor or a pH sensor. The sensor may also be an ultrasonic transducer, a magnetic resonance imaging coil, an optical sensor or lens, an optical coherence tomography sensor or a mechanical excitation and force transducer for elastography imaging. The device may be used to grasp and deliver a patch or film into an orifice or a lumen. The patch may contain tissue, drugs, growth factor, cells or any combination thereof. The openings may be used to form a seal around an area of tissue.

Another embodiment disclosed herein is a tissue gripping device for use in association with a suction device, comprising: a hollow member having a distal end and a proximal end; a head, having a substantially convex contact face and an inlet, the inlet being attachable to the distal end of the hollow member; the substantially convex contact face having at least one opening, the at least one opening being in flow communication with a suction device through the hollow member; and a control device attachable to the proximal end of the hollow member.

Another embodiment disclosed herein is a clip for use in association with a clip applying apparatus, wherein the clip is flexible, substantially bent or curved in stable equilibrium position and can be loaded into a substantially elongate clip cartridge such that the clip's shape in loaded configuration is substantially more elongate than the clip's shape in stable equilibrium position. The clip may have a sharpened tip, and may be rigid, elastic or super elastic. The clip may be composed of shape memory alloy, shape memory polymer, biodegradable polymer, nitinol, stainless steel, titanium or cross-linked polyurethane. The cross sectional shape of the clip may be circular, rectangular or flat.

A further understanding of the functional and advantageous aspects of the present disclosure can be realized by reference to the following detailed description and drawings.

### BRIEF DESCRIPTION OF THE DRAWINGS

Embodiments of the present disclosure will now be described, by way of example only, with reference to the drawings, in which:

FIG. 1 shows the device attached to an existing endoscope.

FIG. 2 shows the detailed view of the distal head of the device.

FIG. 3 shows a dual suction chamber head with a tool channel.

FIG. 4 shows a method of translation, rotation, and pitching of the head using the suction tubes and tool channel.

FIG. 5 shows a method of translation, rotation, and pitching of the head using a suction tube and three cables.

FIG. 6 shows the attachment pitching in series and parallel with an articulating endoscope.

FIG. 7 shows the translation of the head over a flexible endoscope using attachments.

FIG. 8 shows the translation of the head over a flexible endoscope using a sheath.

FIG. 9 shows the profile of the head on an endoscope with respect to a tubular organ.

FIG. 10 shows a method of collapsing and expanding the head using hydraulic or pneumatic ribs.

FIG. 11 shows a method of using expanding and collapsing bladder to expand a suction cup around a suction hole.

FIG. 12 shows a method of using two expanding and collapsing bladders to expand to a suction cup around a suction hole and then pinch the suctioned tissue.

FIG. 13 shows the two modes of operation of micropillars

FIG. 14 shows the application of a superelastic, self-closing clip using the suction grasping as counter traction.

FIG. 15 shows several embodiments are sequential clip loading

FIG. 16 shows a possible manual handle.

FIG. 17 shows two devices attached to a single endoscope.

FIG. 18 shows a head with built in lenses.

FIG. 19 shows a head with attachable devices.

FIG. 20 shows a head with multiple tool channels.

### DETAILED DESCRIPTION

As required, embodiments of the present disclosure are disclosed herein. However, the disclosed embodiments are merely exemplary, and it should be understood that the present disclosure may be embodied in many various and alternative forms.

The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art. For purposes of teaching and not limitation, the illustrated embodiments are directed to a tissue gripping device.

As disclosed herein, the term ‘rigid members’ means a member that does not deform significantly under normal loading for its operation. ‘flexible members’ means that the member deforms significantly in bending but not axial; it may or may not deform significantly in torsional loading. ‘cables’ deform significantly in all loading except axial extension. Finally, ‘cords’ deform significantly in all loadings (axial, bending, torsion).

As disclosed herein the ‘clip’ is any device which joins tissue by use of normal force or the frictional force derived from application of normal force.

As disclosed herein, “superelasticity” is a process where the elastic energy is stored in a solid-to-solid phase change in addition to standard elastic effect where elastic energy is stored in the bonds of the material.

As disclosed herein, “shape memory” is a process where a material can be deformed in a non-elastic, but non-permanent, fashion thereby giving the illusion of permanent deformation. The material may then regain its original form by an application of another stimulus such as, but not limited to, heat, cold, chemical, electrical, or magnetism but excluding deforming into the original shape.

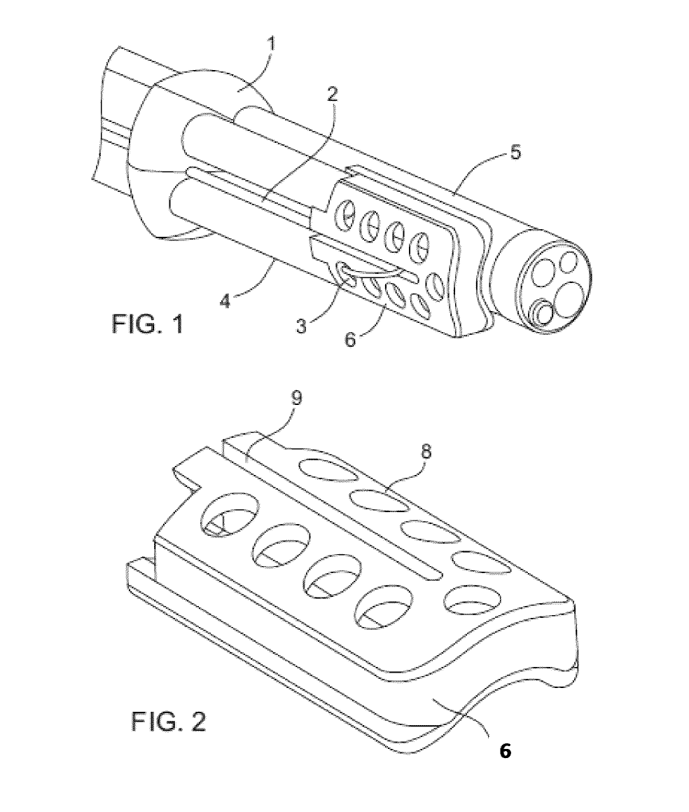

As shown in FIG. 1, the device is comprised of one or more distal heads 6, elongated flexible, rigid, or some combination thereof members 4, and control units. The device can operate as a removable or permanent attachment to the side of an existing endoscope 5, and can be integrated into the endoscope or operate independently of the endoscope. In the embodiment where the device is attached to an existing endoscope 5, the device may additionally comprise one or more attachments 1 to hold the device to the endoscope 5.

As shown in FIG. 2, in one embodiment, the head 6 is a hollow structure with openings 8 on the side facing the tissue. The head 6 is connected to negative pressure by the members 4 in order to pull tissue against the tool using the openings 8. The tool can have one or more chambers 7 to allow separate application of negative pressure to different openings or sets of openings. The negative pressure gripping allows the fixation of tissue and provides gripping force in both the direction perpendicular to the side of tool with the openings 8 and the directions parallel to the side of tool with the openings 8. Furthermore, a tool channel 9 is shown.

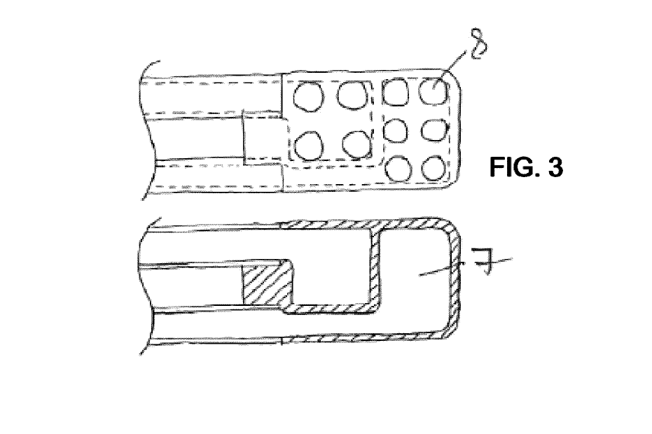

As per the example in FIG. 3, two separate chambers 7 can be used to grasp on edge of the wound, pull into the other edge of the wound, and grasp said other wound edge thereby holding that wound closed. Additionally, the holes 8 can be a mixture of sizes and/or shapes. In one embodiment, large holes are used in the center of the pattern in effect greater perpendicular grasping force and smaller holes are used on the periphery to guard against sudden loss of grasping force. Large holes pull tissue into the hole which creates normal force when pulling parallel to the surface; however, when the seal is broken on a large hole a steep drop in pressure occurs which causes the head to lose grip on the tissue.

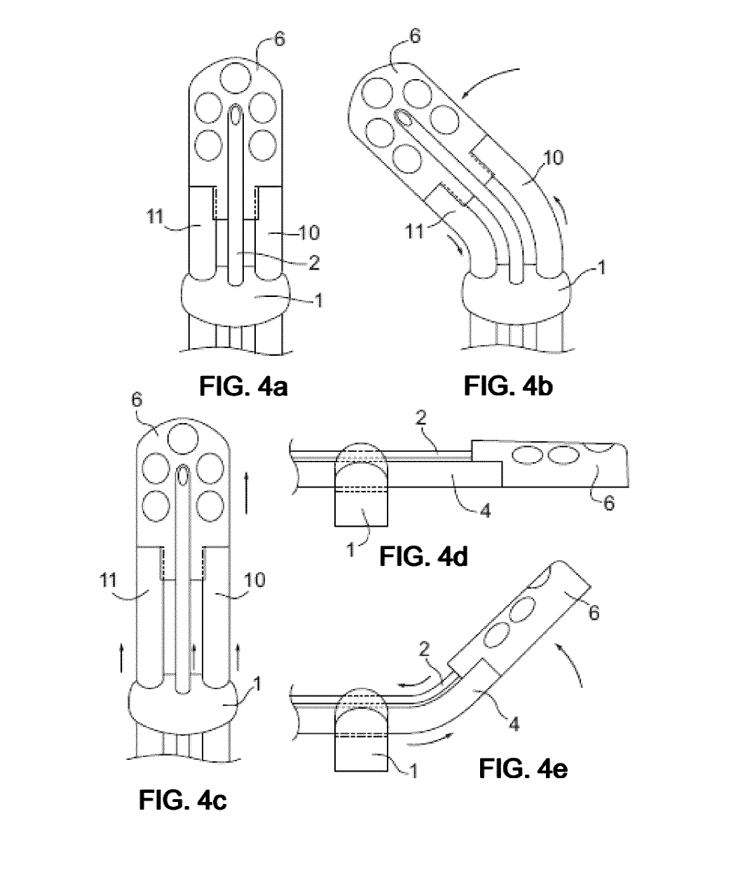

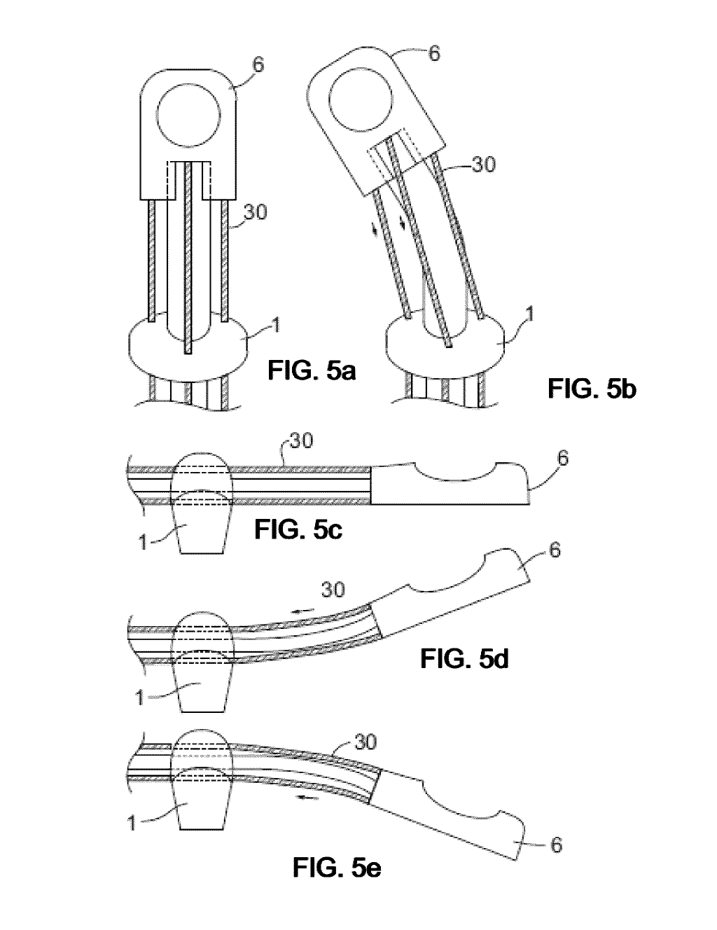

As demonstrated in FIG. 4 and FIG. 5, the head(s) 6 may or may not have the ability to rotate, translate, or pitch axially with respect to each other, the endoscope or the attachment 1. Rotation, translation, or pitching can be accomplished by, but not limited to, mechanical actuation such as pushing and pulling flexible elongated members such as, but not limited to, the elongated members 4, 10 or 11, tool channel 2, added members, expanding pneumatic or hydraulic chambers which may or may not have mesh or flexible reinforcement to direct the deformation, having a central flexible members with pull cables, or some combination thereof where the function members (suction, irrigation, tool access, etc.) may or may not contain or be one of the actuating members. In the embodiment where two flexible vacuum tubes and one flexible tool channel are used (FIG. 4a&e), the right vacuum tube 11 is translated distally and left vacuum 10 tube is translated proximally with the tool channel 2 being kept at neutral translation between the two vacuum tubes in order to rotate the head 6 counterclockwise with the opposite motion accomplishing clockwise motion (FIG. 4b). To achieve translation all three members 11, 10, 2 are translated equally (FIG. 4c). Finally, to pitch the head 6 upward, the tool channel 2 is translated proximally while the vacuum tubes 4 translate distally (FIG. 4d). Combinations of these procedures can be used to achieve combinations of movements. As shown in in another embodiment, a flexible vacuum tube and three cables 30 are used (FIG. 5a). Rotation is accomplished by pulling of the cable on the side which the head is rotating toward and the central cable (FIG. 5b). Pitching up is accomplished by pulling the central cable (FIG. 5c) and down is accomplished by pulling the other two cables (FIG. 5d). Finally, translation is accomplished by translating the flexible vacuum tube (FIG. 5e).

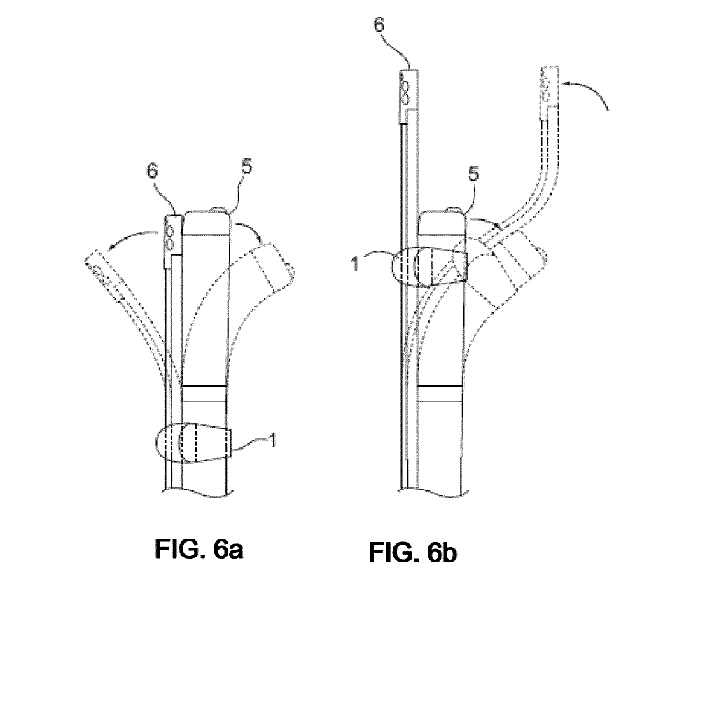

As shown in FIG. 6, the device may also operate with an articulated endoscope 5 and the device can rotate and pitch separately (FIG. 6a) or in addition (FIG. 6b) to the endoscope articulation with respect to an attachment 1. Additionally, superelastic tubing or a spring core with or without a membrane can be used for the tool channel 2 or flexible members to increase flexibility.

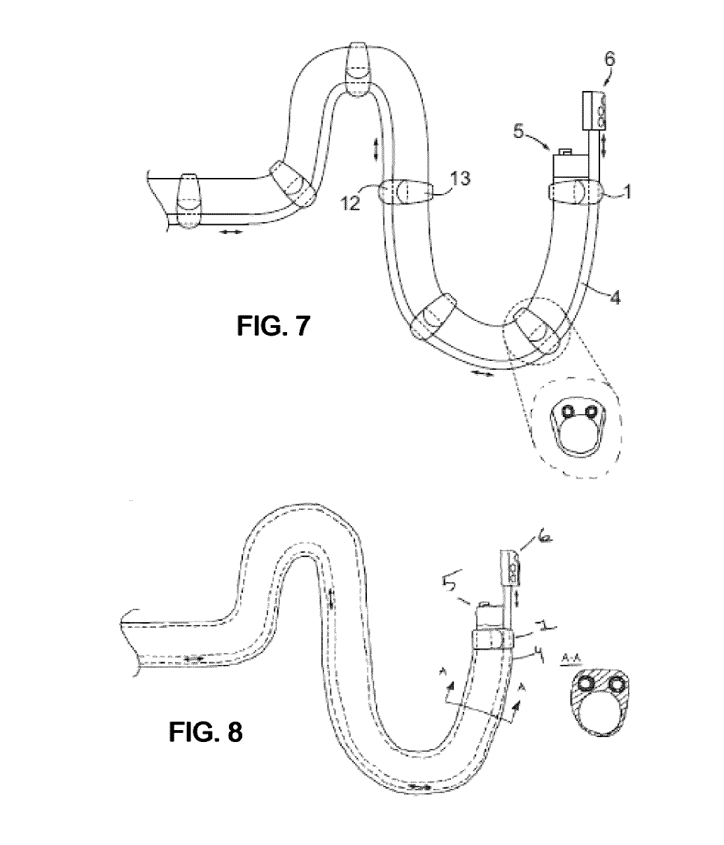

As per the examples given in FIG. 7 and FIG. 8, in the embodiment where the device head 6 is attached to an existing endoscope 5, one or more attachments 1 can be affixed in any non-destructive method as such, but not limited to, clipping, clamping, magnetism, non-permanent adhesion, or sliding over. The attachments 1 can, but are not limited to, a number of clips or loops 12, 13 (FIG. 7), a sheath which fits over the endoscope (FIG. 8), or a tacky silicone rubber. Additionally, the attachments may not actually attach to the endoscope and instead be guides which keep the device properly aligned to the endoscope such that the organ may hold the tool against the endoscope.

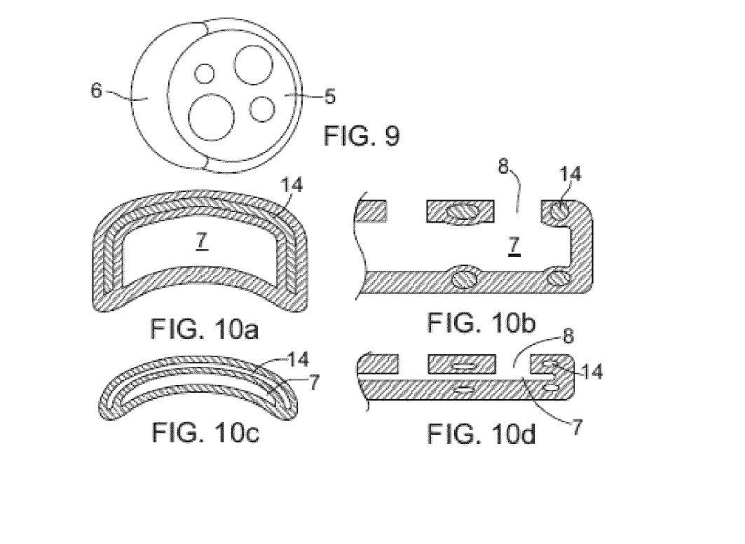

As demonstrated in FIG. 9, the face of the head 6 facing the tissue is specially shaped to match the organ which the tool is intended to grasp and is rigid when in use. The face of the head facing the tissue can also be selected based on patient age and/or preoperative imaging such as, but not limited to, CT, MRI, capsule endoscopy, ultrasound, or some combination or fusion thereof. Age is important beyond sizing as younger tissue is more fragile and the suction grasping must be made more gentle by adjusting factors such as, but not limited to, pressure, surface grasping softness, and hole edge fillet radius. Flexible faces require that they be forced into the surface to deform it to the shape of the organ and can be peeled off by pulling up an edge thereby breaking the suction grasping; therefore, the rigid face of our tool allow more responsive and more reliable suction grasping versus a flexible face. Additionally, in the embodiment where the device is attached to an endoscope 5, the side facing the endoscope is specially shaped to match the endoscope which prevents pinching of tissue and provides economy of space.

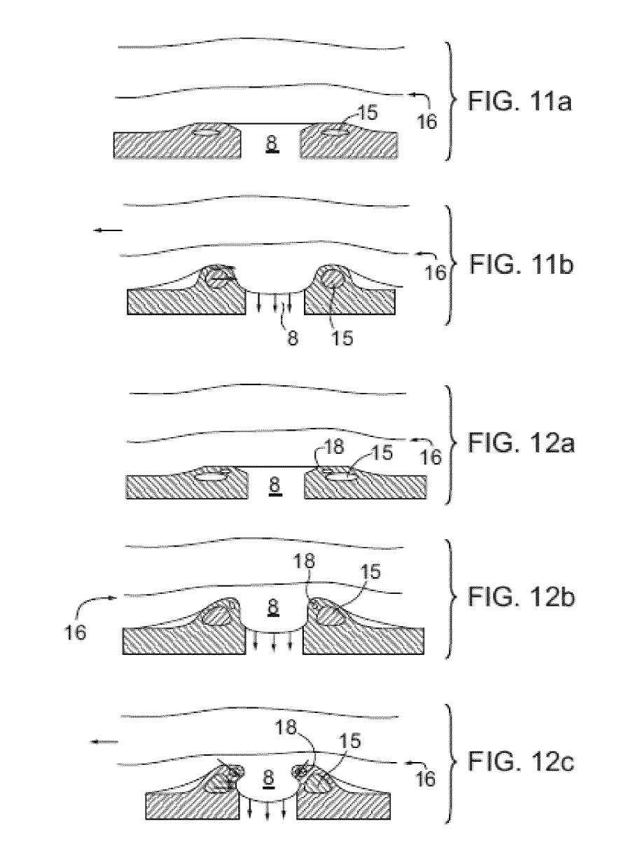

As shown in the examples in FIG. 10, FIG. 11, and FIG. 12, in another embodiment, the head 6 can be expanded and collapsed. This can be accomplished by, but is not limited to, mechanical actuation, such as pulling or pushing a four-bar mechanism, hydraulically or pneumatically, such as expanding or contracting a bladder to force the same four-bar mechanism, or using material properties, such as using body heat to expand shape memory polymer. Additionally, the body could be made partly or fully soft and filling of a bladder 14 creates the structure (FIG. 10). In this example, the head is collapsed for insertion through smaller organs such as the esophagus (FIG. 10c&d) and then hydraulic or pneumatic ribs 14 are inflated to expand the head into a rigid structure once the head reaches a larger organ such as the small intestine (FIG. 10a&b). Finally, the expanding or collapsing of the head can be used for tissue manipulation. In one embodiment, a bladder 16 placed around a suction hole 8 (FIG. 11a) expand outward thereby creating a suction cup (FIG. 11b). In a more complex form of this embodiment (FIG. 12a), there is yet another bladder 17 under the first 16 such that, after the first is expanded to form the suction cup and suction is applied to the hole which the bladders surround (FIG. 12b), the second is expanded to clamp the suction cup onto the tissue 15 (FIG. 12c).

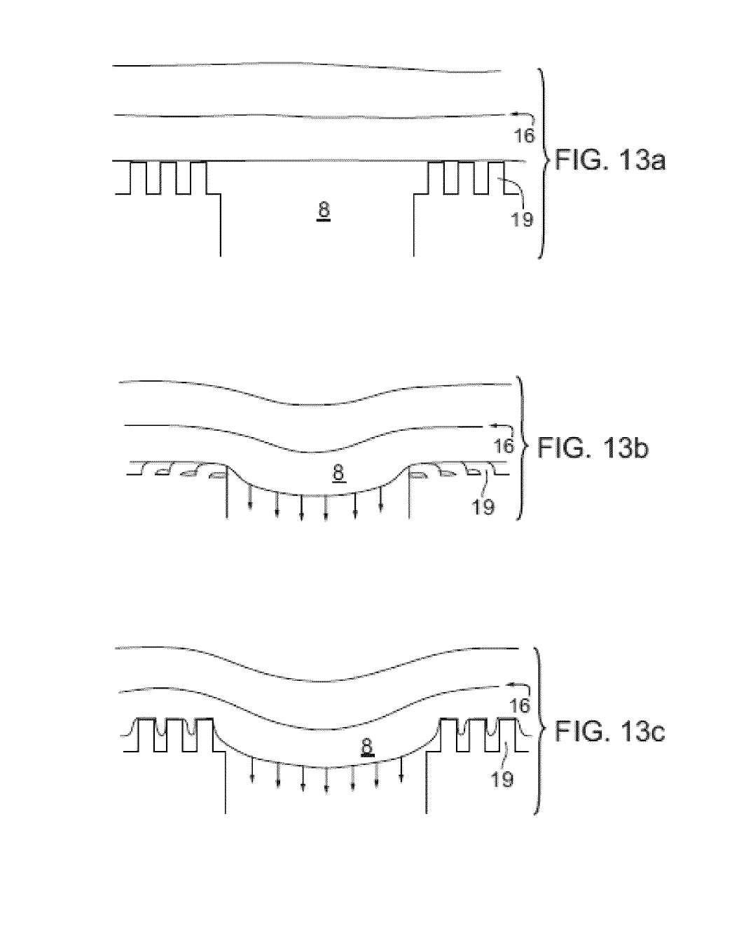

As shown in FIG. 13, micropillars 18 on the tissue contacting surface present little surface area when little perpendicular force is applied towards the opening 8 (FIG. 13a) which reduces friction but when perpendicular force is applied the micropillars flatten (FIG. 13b) and increase their surface area thereby drastically increasing friction. This simultaneously allows smoother insertion and higher gripping strength. Additionally, stiffer micropillars 18 do not flatten when perpendicular force is applied (FIG. 13c). In this fashion, they operate the same as softer micropillars 18 at low perpendicular force but at high perpendicular force the tissue conforms over the tissue 16 which creates parallel holding force party through normal force, which is unaffected by lubrication, rather than frictional force. The degree to which the each effect acts can be approximately determined by the free-fixed end column buckling equation which is dependent on the elastic modulus of the material, bending moment of the pillar, and the height of the pillar. The pillar 18 can be of any cross-sectional geometry such as but not limited to circular, groves, elliptical, or rectangular.

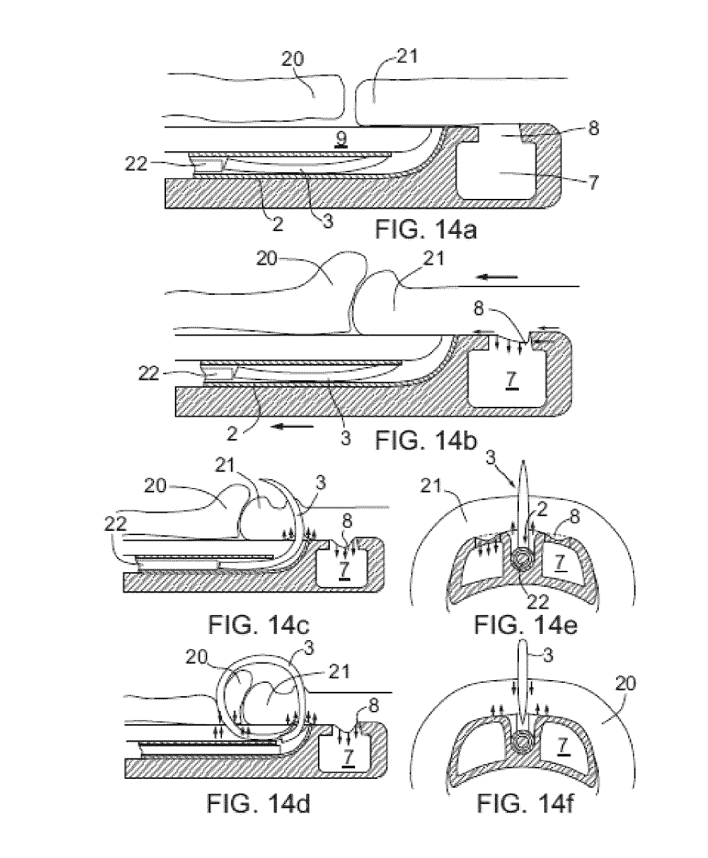

As one method is demonstrated in FIG. 14, the aforementioned suction acts as counter traction which allows clips 3 such as, but not limited to, superelastic nitinol, elastic polyurethane, or rigid stainless steel to be placed in a looping motion. In one method of clip application, clip application is accomplished as follows. First, the head 6 is advanced past the wound site with the superelastic clip 3 bent flat inside the tool channel 2 (FIG. 14a). Next, the head 6 uses suction to grasp the distal wound edge 20 and pull it parallel to surface of the head 6 and into the other wound edge 19 (FIG. 14b). Then, the superelastic clip 3 is forced out of the tool channel 2 by a ram rod 21 and it assumes its relaxed state of the circle. The end of the channel is curved with the inside of the curve removed to orient the clip to the tool trench and to prevent the clip from getting caught by the channel respectively (FIG. 14c&d). The far wound edge 20 is held in place by surrounding suction holes 8 thereby providing the clip 3 the counter traction required to pierce the tissue. Finally, as the clip 3 curls back, the proximal wound edge 19 is pinned between the clip 3 and the trench 9 in which the tool channel 2 is placed. Additionally, the suction holes 8 can provide counter traction but are not essential (FIG. 14e&f).

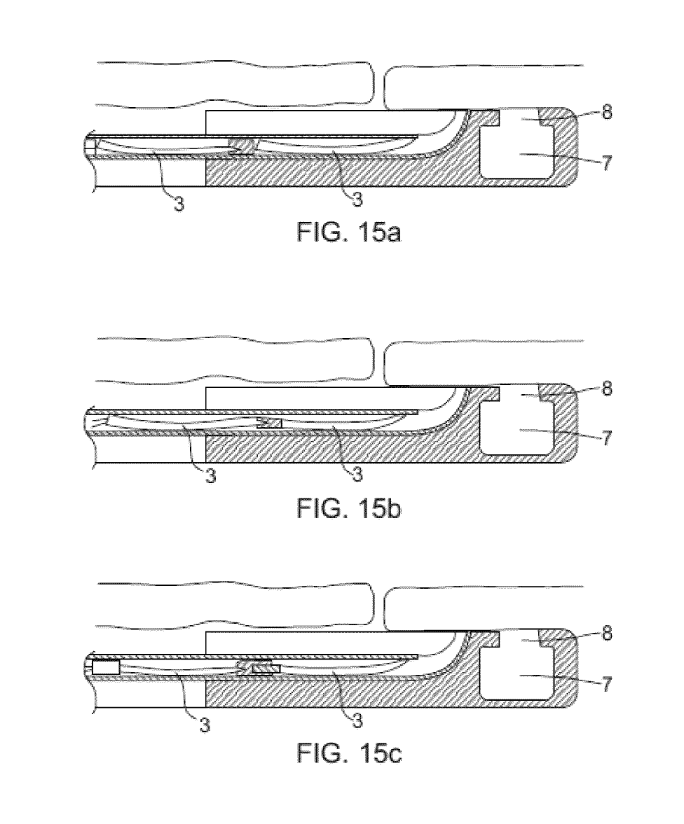

As multiple solutions are given in FIG. 15 to enable the function, clips can be loaded in series in these cartridges or in the tool channel. They require a safety cap 38 to protect the sharp tip of the clip from damage and causing damage and to reduce friction. In one embodiment, the cap is designed such that it falls off when the tip of the clip exits the tool channel and can be made of the biodegradable and/or biocompatible compound. One way of achieving the cap is to have the tip pinned inside a split cylinder which is sized to fit the cartridge channel or tool channel depending on the embodiment (FIG. 15a). Additionally, the cap can be integrated into the next clip. As examples, the cap can be affixed to the end of the clip (FIG. 15b) or can be integral and manufactured into the clip (FIG. 15c).

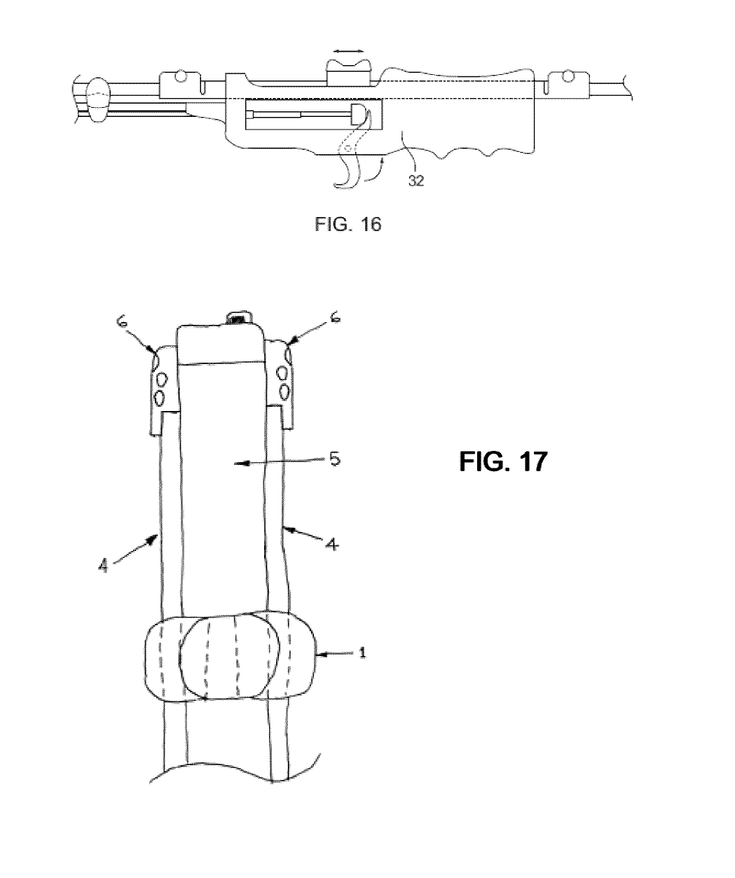

As demonstrated in the manual handle example in FIG. 16, the control device 32 can be any embodiment that allows control of the functions present in the heads 6. They can be manual, electronic, robotic, or any combination thereof. They may also, but are not limited to, contain sensors, attach to the endoscope, or be integrated into the endoscope control handle.

These attachments 1 can facilitate the aforementioned motions over a flexible path with any number of bends by providing a short enough distance between attachments 1, stiff enough tubing 4, and smooth enough motion such that the tubes do not buckle when translated. In this example, there are many attachments 1 placed along the length of the endoscope 5. The attachments have a high friction, elastic strap 13, such as silicone, to hold the attachments 1 in place on the endoscope and a low friction rigid body 12, such as PTFE or UHMWPE, to allow smooth translation of the tubes.

The suction can be derived from any source such as, but not limited to, medical wall suction, hand pump, or pump.

The members 2 are hollow and provide the negative pressure and the tool channel to the head 6 and can be flexible or rigid. In addition to negative pressure and tools, any other fluid, gas, or plasma can be delivered through the channels to perform operation such as, but not limited to, intubation, irrigation, insufflation, or anesthesia. The members 2 may also be made transparent to provide less obstruction of view.

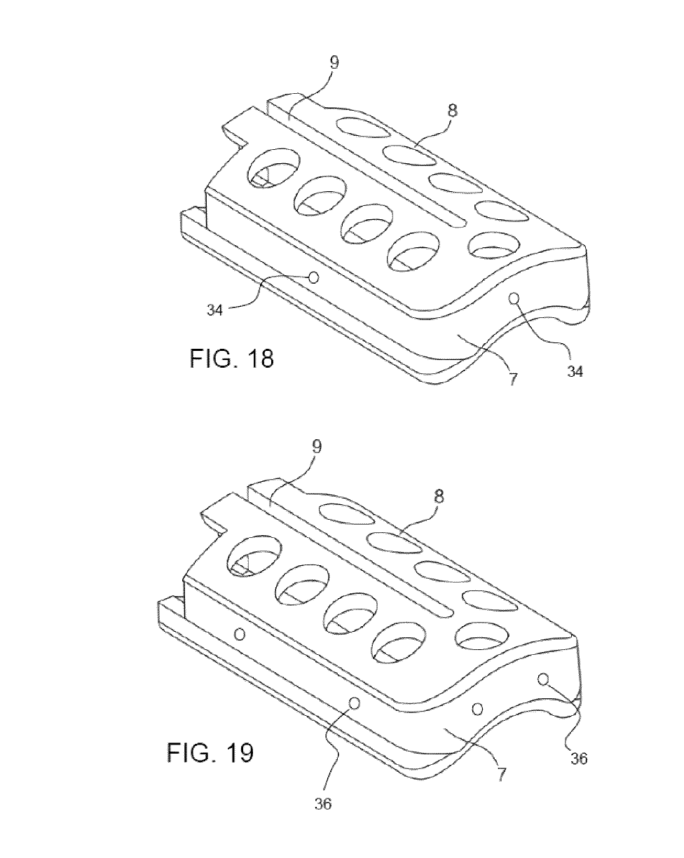

There may be openings placed on the head(s) in order to facilitate viewing during an operation. Also, as shown in FIG. 18 lenses 34 may be located on the head to magnify the site of interest. Additionally, the head may be transparent to allow for increased vision during surgery. The openings, lenses, or transparent body can be placed for better viewing of the tool hole exits as well. These lenses may mate with the endoscope to provide better vision of the tissue being grasped thereby extending the endoscope lens assembly.

The surface of the head 6 with holes in it that faces the tissue can be modified to increase or decrease grasping force. This is accomplish by, but not limited to, knurling, abrading, particle embedding, the aforementioned micropillars, hydrophobicity, hydrophilicity, or some combination, mixture, or arrangement thereof. Knurling, abrading, and particle embedding increase the friction when pulling parallel to the suction face. Hydrophobicity can be used to force mucus and bodily fluid away from the surface thereby, but not limited to, keeping the surface clean for viewing through a transparent body, cleaning the esophageal wall when force into it, reducing perpendicular force on tissue due to loss of fluid viscosity, and/or increasing friction. Hydrophilicity can be used to hold bodily fluids to the surface thereby, but not limited to, increasing lubrication, reducing friction, and increasing perpendicular force on tissue by increased fluid viscosity.

As shown in FIG. 19, the head 6 can also have devices 36 such as sensors or imaging modalities integrated into it. These sensors can potentially, but are not limited to, measure contact, conductance, pressure, force, and/or capacitance. An example of the use of pressure and force sensors, is determining if the bulk force being applied to the tissue is locally concentrated enough to damage cells. Another example of the use of pressure and force sensors is to palpate the tissue by using the suction system in an oscillatory fashion and using the pressure/force distribution to determine the stiffness of the tissue; this imaging method is known as elastography. The head 6 may also have ultrasonic transducers, magnetic resonance imaging coils, optical sensors or lenses, photoacoustic imaging apparatus, or optical coherence tomography integrated. As an example, OCT or ultrasound could be used to determine the thickness of the esophageal wall and layers and to determine the composition of polyps and sores. The sensors measurements and imaging modalities may also be graphically overlaid with the images produced by the endoscope or may be overlaid with each other.

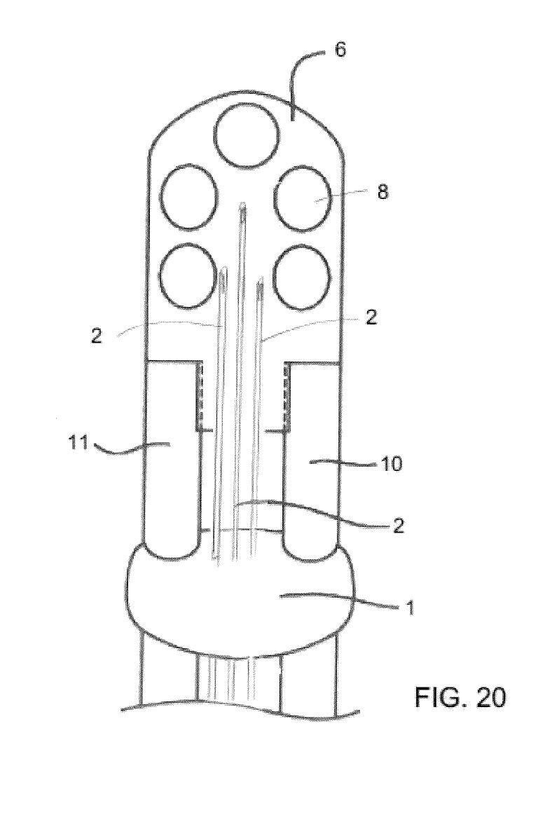

As shown in FIG. 20, the head 6 has one or more tool channels 2. These channels facilitate standard endoscopic tools such as, but not limited to, electrocautery, biopsy needles, ligation loops, snares, ablation, and injection therapy. Additionally, the tool channel can accommodate superelastic clips composed of materials such as nitinol. The tool channel 2 has an opening facing the tissue and may or may not have an opening on the opposing side to facilitate viewing of the operation the tool channel. The suction holes act as counter traction for tools requiring force such as, but not limited to, biopsy, injection, and surgical clip application. The suction pattern and force requiring tool must be specially designed for one another such that the suction is capable of providing the specific amount and direction of force required for the tool. The required force of the tool and force capability of the suction pattern can be determined by, but is not limited to, anatomy specific finite element analysis (FEA) model, animal tissue testing, or existing literature. For the example of a clip, the needle tip geometry, surface finish, and diameter and suction hole size, pressure, and hole geometry can be selected, but is not limited to these variables, such that the suction can hold the tissue in the direction perpendicular to the tissue grasping surface while the clip is being applied. In the example of biopsy, it is advantageous to perform at an angle oblique to the suction surface. Thus, the perpendicular and parallel force need to be designed which can be achieved by different mechanisms. As examples, micropillars increase parallel force but do little for perpendicular force whereas increasing total suction opening size affects both directions.

In the embodiment where surgical clips are applied through the tool channel, the surgical clips can be rigid, elastic, or superelastic and these clips may or may not bend back on themselves. In the case of elastic and superelastic clips, the clip is deformed before application and springs back into its relaxed circular shape. In the case of rigid clips, the clip must be permanently deformed into a circular shape as it exits the tool channel. Bending back on themselves prevents tissue for escaping and the wound closure from opening. Clips can be composed of, but not limited to, superelastic metal such as nitinol, stainless steel, titanium, shape memory metal such as nitinol, shape memory polymer such as cross-linked polyurethane, biodegradable polymer, shape memory, biodegradable polymer, ceramic, silicone, or any combination, composite, laminate, coating, embedding, patterning, alloy, polymerization, or arrangement thereof.

The ram rod can be actuated in any way including, but not limited to, manual pushing, hydraulic, pneumatic, piezoelectric, electric motor, heat expansion, or combination thereof.

This method provides a suture-like, protrusion-less closure whereas existing endoscopic surgical clips protrude from the organ surface. The openings 8 may be arranged in such a way that they surround or mostly surround the tool channel 2 exit in order to provide better counter traction. Additionally, smaller holes may be placed around the tool hole to provide a more gradual loss of suction as was elaborated on earlier for suction tissue manipulation.

The clips are generally tipped at the distal end and flat or blunt at the proximal end to allow puncturing and ram rod force respectively. The end of the clip can, however, be of any profile such as, but not limited to, blunt, conical, beveled, diamond, flat. The tip or clip may also be treated or coated with, but not limited to, any combination of drugs, polishing, abrading, oxide, indicator, polymer, carbide, silicone, hydrogel, biologic, or cells. As examples, endothelial cells proliferate best at around a 2400 grit surface, cells differentiate preferentially based on the hardness of their surroundings so a coating matching the hardness of the organ the clip is embedded in is preferable, the titanium oxide coating on nitinol is known to be extremely bioinert, and growth factors can be used to control the wound healing process thereby speeding or improving wound healing. Finally, additional features may be manufactured onto the clip such as, but not limited to, diamond or carbide tipping or rubber or plastic ends.

The clip(s) can be loaded into hypodermic tube cartridges that are then loaded into the tool channel. This allows the clips to be loaded from the proximal end of the tool and allows empty cartridge to be quickly exchanged with loaded ones without removing the tool or endoscope.

Beyond, the aforementioned curved tool channel exit orientation can also be accomplished by partial flattening of the tool channel and/or cartridge to create an elliptical profile. In this embodiment, the elasticity of the clip(s) forces it to align with the longer direction of the ellipse. This method only guarantees alignment along this axis leaving two possible orientations; thus, the channel must be marked to indicate which of these two orientations the clip(s) are in. Additionally, the clip can be manually orientated and then the surgeon can impart orientation manually or by forced orientation of the cartridge such as, but not limited to, keying. Finally, any method which ensures the orientation of the clip(s) may be used.

Additionally, these holes 8 can be placed around the tool channel 2 exit and a sealing ring composed of soft material such as, but not limited to, silicone, neoprene, polyurethane, or Buna-N may or may not also be placed to seal around a section of tissue to allow localized delivery of collapsed mesh structures, fluid, gel, gas, plasma, drugs, growth factors, cells or any combination thereof. For example, this facilitates localized drug delivery, plasma ablation, and delivery of cells suspended in hydrogel with growth factors for regenerative medicine.

The device can be used to perform anastomosis by one or more separate heads. As an example, three heads can be used in the following manner: one to grasp one side of the wound, another to gasp the other side of the wound, and the third to provide counter traction for suture placement. Additionally, one or more heads can be used in a fashion such that they can traverse the small intestine. As an example, if two heads are used: negative pressure is applied to head one, head two is advanced, negative pressure is applied to head two and removed from head one, head two is retracted and pulls the small intestine down with it, head two has negative pressure removed, and then the process is repeated.

In a preferred embodiment for performing traversal of the small intestine, two heads are used where both have a single suction chamber and one or both have the ability to translate. As demonstrated in FIG. 17, two heads 6 may be slidably attached with an attachment 1 to a single device such as an endoscope 5. In one embodiment, the heads may alternate gripping tissue while the other navigates an orifice, or tubular viscera. Additionally, the aforementioned bladder expansion is used in order to pass through the smaller esophagus and then expand to match the small intestine as determined by anatomy, age, and/or pre-operative imaging. Next, the soft micropillar array is preferred with this embodiment as it allows smoother traversal through the esophagus and less trauma when advancing the heads to grasp tissue in the small intestine. Finally, the suction tubes are spring core with a polymer membrane to allow increased flexibility. The two tissue manipulation heads can also be used in a diagnostic fashion by palpating tissue individually or by use of differential motion. As an example of palpation, if a surgeon wishes to non-invasively ascertain the contents of a polyp he can pull on the tissue surrounding the polyp; if the polyp deforms it is filled with fluid such as blood or puss but if it does not then it is solid and may be cancerous.

In another preferred embodiment for anastomosis of the esophagus, two heads are used where the first head is attached to the endoscope by attachments and the second head is coupled to the endoscope but is permitted to freely rotate about the axis of the endoscope. Additionally, the first head has a single chamber attached to all suction holes, a tool channel loaded with the previous disclosed superelastic clip applying apparatus, and the ability to translate, rotate, and pitch by using the two suction tubes and one tool channel and has the ability to roll with the endoscope. The second head has two chambers, no tool channel, and the ability to translate and rotate by use of the two suction tubes. This embodiment operates as follows. Initially, the first head is used to retrieve the distal lumen and pull it back towards the endoscope. Secondly, the distal chamber of the second head grasps the distal lumen, pulls it into the proximal lumen, and then the proximal chamber of the second head grasps the proximal lumen thereby temporarily coupling the organ. Then, the first head is used to clip the lumens together as per the earlier example of clip application. Finally, the endoscope is then rolled by an increment thereby leaving the second hand in place and rolling the first head along the lumen interface. The clip application is then repeated. This process is repeated until the lumens are anastomosed. As a variation in use, the second step may be changed to, “the proximal chamber of the second head grasps the proximal lumen, pushes it towards the distal lumen, and then the distal chamber grasps the distal lumen thereby temporarily coupling the organ”. This alternative is useful where one lumen is fixed in place by connective tissue or other anatomy such as, but not limited to, nerves, blood vessels, bone, or other organs.

In a preferred embodiment for puncture or fistula closure, a single head is used with two suction channels with one suction chamber and one tool channel. The tool channel is used with the superelastic clip applying apparatus that was previously disclosed. The tool is attached to an endoscope and has the ability to translate by use of the suction tubes and tool channel and can translate and rotate by attachment to the endoscope. The tool operates as per the example given for clip application. It can also provide tissue manipulation for finding punctures by pulling on tissue to open up said punctures or fistula for easy detection.

In another embodiment of the present disclosure, the device may be inserted through an incision in the body as opposed to through a natural orifice.

As used herein, the terms “comprises”, “comprising”, “includes” and “including” are to be construed as being inclusive and open ended, and not exclusive. Specifically, when used in this specification including claims, the terms “comprises”, “comprising”, “includes” and “including” and variations thereof mean the specified features, steps or components are included. These terms are not to be interpreted to exclude the presence of other features, steps or components.

The foregoing description of the preferred embodiments of the present disclosure has been presented to illustrate the principles of the disclosure and not to limit the disclosure to the particular embodiment illustrated. It is intended that the scope of the present disclosure be defined by all of the embodiments encompassed within the following claims and their equivalents.

Therefore what is claimed is:

1. A tissue gripping device for use in association with a clip having a distal end and a suction device, comprising:
a hollow member having a distal end and a proximal end;
a head, having a contact face and an inlet, the inlet being attachable to the distal end of the hollow member;
a tool channel having a distal end and a proximal end;
the contact face having at least one opening, the at least one opening being in flow communication with a suction device through the hollow member;
a generally elongate tool housing formed in the head and configured to receive the clip wherein the tool housing is attachable to and in flow communication with the distal end of the tool channel;
a tool trench formed in the head configured to receive therein the distal end of the clip; and
a control device attachable to the proximal end of the hollow member and operably attachable to the proximal end of the tool channel, whereby the clip is ejected responsive to actuation by the control device from the tool channel and into the tool trench.

2. The tissue gripping device as claimed in claim 1, wherein the clip is loaded into the tool housing through the proximal end of the tool channel.

3. The tissue gripping device as claimed in any one of claim 1 or 2, wherein the clip has a loop shape in stable equilibrium position.

4. The tissue gripping device as claimed in any one of claims 1 to 3, wherein the clip is composed of elastic material, super-elastic material or shape memory material.

5. The tissue gripping device as claimed in claim 4, wherein the clip is composed of self-closing, super-elastic nitinol.

6. The tissue gripping device as claimed in any one of claims 1 to 5, wherein the tool housing has an embedded ejection mechanism for ejecting the clip.

7. The tissue gripping device as claimed in claim 6, wherein the embedded ejection mechanism is mechanically, electro-dynamically, pneumatically or hydraulically actuated from the control device.

8. The tissue gripping device as claimed in claim 7, wherein the head further comprises an inflatable bladder in flow communication with a bladder pump.

9. The tissue gripping device as claimed in claim 8, wherein the inflatable bladder can be inflated or deflated with the bladder pump such that size dimensions of the head can be increased or decreased, responsive to the bladder pump.

10. The tissue gripping device as claimed in claim 8 or 9, where in the inflatable bladder can be inflated or deflated with the bladder pump such that size dimensions of the at least one opening can be increased or decrease, respectively.

11. The tissue gripping device as claimed in any one of claims 8 to 10, wherein the inflatable bladder is inflated or deflated hydraulically or pneumatically.

12. The tissue gripping device as claimed in any one of claims 1 to 11, wherein the hollow member is flexible.

13. The tissue gripping device as claimed in any one of claims 1 to 12, wherein the tissue gripping device further comprises at least one flexible member having a distal end and a proximal end wherein the distal end it attachable to the head such that relative translation between the hollow member and at least one flexible member rotates the head.

14. The tissue gripping device as claimed in claim 13, wherein an attachment is slidably attached to the hollow member, the at least one flexible member and the tool channel such that the head can rotate or translate with respect to the attachment.

15. The tissue gripping device as claimed in claim 14, further including a second tissue gripping device with a second device member wherein the second device member is slidably attached to the attachment.

16. The tissue gripping device as claimed in any one of claims 1 to 15, wherein the hollow member is reinforced with a spring core or a superelastic tube.

17. The tissue gripping device as claimed in any one of claims 1 to 16, wherein at least one chord, rope, string or wire is attachable to the head such that tension can be applied to rotate the head with respect to the hollow member.

18. The tissue gripping device as claimed in any one of claims 1 to 17, wherein the contact face has a convex shape.

19. The tissue gripping device as claimed in claim 18, wherein the convex shape has a radius of convexity similar to a radius of concavity of a natural orifice or a natural lumen.

20. The tissue gripping device as claimed in any one of claims 1 to 19, wherein the hollow member is a conduit in flow communication with the at least one opening.

21. The tissue gripping device as claimed in claim 20, wherein the conduit is in flow communication with the suction device.

22. The tissue gripping device as claimed in claim 21, wherein the suction device is one of a pump, a vacuum and a pump and vacuum.

23. The tissue gripping device as claimed in any one of claims 1 to 22, wherein a surface of the contact face is treated with microgrooves.

24. The tissue gripping device as claimed in claim 23, wherein the microgrooves are stiff micro-pillars extending outwardly from the contact face.

25. The tissue gripping device as claimed in claim 24, wherein the microgrooves are soft micro-pillars extending outwardly from the contact face such that the soft micro-pillars deform under sheering and normal force.

26. The tissue gripping device as claimed in any one of claims 23 to 25, wherein the surface of the contact face is treated with a hydrophilic, hydrophobic, knurled or abraded coating.

27. The tissue gripping device as claimed in any one of claims 1 to 26, wherein the head is rigid.

28. The tissue gripping device as claimed in any one of claims 1 to 27, wherein one of at least one lens, window and fiber optic cable is built into the head.

29. The tissue gripping device as claimed in any one of claims 1 to 28, wherein the head is constructed from transparent or translucent material.

30. The tissue gripping device as claimed in any one of claims 1 to 29, wherein the contact face comprises a plurality of openings of different sizes such that smaller openings are placed proximate to the edge of the contact face.

31. The tissue gripping device as claimed in any one of claims 1 to 30, wherein the tissue gripping device is attachable to an endoscope, a drug administering device, an electrode or a medical retractor.

32. The tissue gripping device as claimed in any one of claims 1 to 31, wherein the head is attachable to a sensor.

33. The tissue gripping device as claimed in claim 32, wherein the sensor is capable of measuring at least one of the following: force, pressure, capacitance, temperature and pH.

34. The tissue gripping device as claimed in claim 33, wherein the sensor is an ultrasonic transducer, a magnetic resonance imaging coil, an optical sensor or lens, an optical coherence tomography sensor or a mechanical excitation and force transducer for elastography imaging.

35. The tissue gripping device as claimed in any one of claims 1 to 34, where the at least one opening is used to grasp and deliver a patch or a film into an orifice or a lumen.

36. The tissue gripping device as claimed in claim 35, wherein the patch or the film contains tissue, drugs, growth factor, cells or any combination thereof.

37. The tissue gripping device as claimed in any one of claims 1 to 36, wherein the contact face can seal an area of tissue.

38. The tissue gripping device as claimed in any one of claims 1 to 36, wherein the tissue gripping device comprises at least a first hollow member and a second hollow member and at least a first opening and a second opening in the contact face, such that the first opening is in flow communication with a first suction device through the first hollow member, and the second opening is in flow communication with a second suction device through the second hollow member wherein the first hollow member is not in flow communication with the second hollow member.

39. A tissue gripping device for use in association with a suction device and a bladder pump, comprising:
a hollow member having a distal end and a proximal end;
a head having a contact face and an inlet, wherein the inlet is attachable to the distal end of the hollow member;
an inflatable bladder, formed in the head, in flow communication with the bladder pump;
the contact face having at least one opening wherein the at least one opening is in flow communication with the suction device through the hollow member; and
a control device attachable to the proximal end of the hollow member.

40. The tissue gripping device as claimed in claim 39, wherein the inflatable bladder can be inflated or deflated with the bladder pump such that size dimensions of the head can be increased or decreased, responsive to the bladder pump.

41. The tissue gripping device as claimed in claim 39 or 40, where in the inflatable bladder can be inflated or deflated with the bladder pump such that size dimensions of the at least one opening can be increased or decrease, respectively.

42. The tissue gripping device as claimed in any one of claims 39 to 41, wherein the inflatable bladder is inflated or deflated hydraulically or pneumatically.

43. The tissue gripping device as claimed in any one of claims 39 to 42, wherein the hollow member is flexible.

44. The tissue gripping device as claimed in any one of claims 39 to 43, further comprises at least one flexible member having a distal end and a proximal end, the distal end being attachable to the head such that relative translation between the hollow member and at least one flexible member rotates the head.

45. The tissue gripping device as claimed in claim 44, wherein an attachment is slidably attached to the hollow member and the at least one flexible member such that the head can rotate or translate with respect to the attachment.

46. The tissue gripping device as claimed in claim 45, further including a second tissue gripping device with a second device member wherein the second device member is slidably attached to the attachment.

47. The tissue gripping device as claimed in any one of claims 39 to 46, wherein the hollow member is reinforced with a spring core or a superelastic tube.

48. The tissue gripping device as claimed in any one of claims 39 to 47, wherein at least one chord, rope, string or wire is attachable to the head such that tension can be applied to rotate the head with respect to the hollow member.

49. The tissue gripping device as claimed in any one of claims 39 to 48, wherein the contact face has a convex shape.

50. The tissue gripping device as claimed in claim 49, wherein the convex shape has a radius of convexity similar to a radius of concavity of a natural orifice or a natural lumen.

51. The tissue gripping device as claimed in any one of claims 39 to 50, wherein the hollow member is a conduit in flow communication with the at least one opening.

52. The tissue gripping device as claimed in claim 51, wherein the conduit is in flow communication with the suction device.

53. The tissue gripping device as claimed in claim 52, wherein the suction device is a pump or a vacuum.

54. The tissue gripping device as claimed in any one of claims 39 to 53, wherein a surface of the contact face is treated with microgrooves.

55. The tissue gripping device as claimed in claim 54, wherein the microgrooves are stiff micro-pillars extending outwardly from the contact face.

56. The tissue gripping device as claimed in claim 55, wherein the microgrooves are soft micro-pillars extending outwardly from the contact face such that the soft micro-pillars deform under sheering and normal force.

57. The tissue gripping device as claimed in any one of claims 54 to 56, wherein the surface of the contact face is treated with a hydrophilic, hydrophobic, knurled or abraded coating.

58. The tissue gripping device as claimed in any one of claims 39 to 57, wherein the head is rigid.

59. The tissue gripping device as claimed in any one of claims 39 to 58, wherein at least one lens, window or fiber optic cable is built into the head.

60. The tissue gripping device as claimed in any one of claims 39 to 59, wherein the head is constructed from transparent or translucent material.

61. The tissue gripping device as claimed in any one of claims 39 to 60, wherein the contact face comprises a plurality of openings of different sizes such that smaller openings are placed proximate to the edge of the contact face.

62. The tissue gripping device as claimed in any one of claims 39 to 61, wherein the tissue gripping device is attachable to an endoscope, a drug administering device, an electrode or a medical retractor.

63. The tissue gripping device as claimed in any one of claims 39 to 62, wherein the head is attachable to a sensor.

64. The tissue gripping device as claimed in claim 63, wherein the sensor is capable of measuring at least one of the following: force, pressure, capacitance, temperature and pH.

65. The tissue gripping device as claimed in claim 64, wherein the sensor is an ultrasonic transducer, a magnetic resonance imaging coil, an optical sensor or lens, an optical coherence tomography sensor or a mechanical excitation and force transducer for elastography imaging.

66. The tissue gripping device as claimed in any one of claims 39 to 65, where the at least one opening is used to grasp and deliver a patch or a film into an orifice or a lumen.

67. The tissue gripping device as claimed in claim 66, wherein the patch or the film contains tissue, drugs, growth factor, cells or any combination thereof.

68. The tissue gripping device as claimed in any one of claims 39 to 67, wherein the contact face can seal an area of tissue.

69. The tissue gripping device as claimed in any one of claims 39 to 67, wherein the tissue gripping device comprises at least a first hollow member and a second hollow member and at least a first opening and a second opening in the contact face, such that the first opening is in flow communication with a first suction device through the first hollow member, and the second opening is in flow communication with a second suction device through the second hollow member wherein the first hollow member is not in flow communication with the second hollow member.

70. A tissue gripping device for use in association with a suction device, comprising:
at least two members each having a distal end and a proximal end, wherein one of the at least two members is a flexible member and wherein one of the at least two members is a hollow member;
a head, having a contact face and an inlet, where the inlet is attachable to the distal end of the hollow member;
the contact face having at least one opening wherein the at least one opening is in flow communication with the suction device through the hollow member; and
a control device attachable to the proximal end of the at least two members.

71. The tissue gripping device as claimed in claim 70, wherein the hollow member is flexible.

72. The tissue gripping device as claimed in claim 70 or 71, wherein the distal end it attachable to the head such that relative translation between the at least two members rotates the head.

73. The tissue gripping device as claimed in claim 72, wherein an attachment is slidably attached to the at least two members such that the head can rotate or translate with respect to the attachment.

74. The tissue gripping device as claimed in claim 73, further including a second tissue gripping device with a second device member wherein the second device member is slidably attached to the attachment.

75. The tissue gripping device as claimed in any one of claims 70 to 74, wherein the hollow member is reinforced with a spring core or a superelastic tube.

76. The tissue gripping device as claimed in any one of claims 70 to 75, wherein at least one chord, rope, string or wire is attachable to the head such that tension can be applied to rotate the head with respect to the hollow member.

77. The tissue gripping device as claimed in any one of claims 70 to 76, wherein the contact face has a convex shape.

78. The tissue gripping device as claimed in claim 77, wherein the convex shape has a radius of convexity similar to a radius of concavity of a natural orifice or a natural lumen.

79. The tissue gripping device as claimed in any one of claims 70 to 78, wherein the hollow member is a conduit in flow communication with the at least one opening.

80. The tissue gripping device as claimed in claim 79, wherein the conduit is in flow communication with the suction device.

81. The tissue gripping device as claimed in claim 80, wherein the suction device is a pump or a vacuum.

82. The tissue gripping device as claimed in any one of claims 70 to 81, wherein a surface of the contact face is treated with microgrooves.

83. The tissue gripping device as claimed in claim 82, wherein the microgrooves are stiff micro-pillars extending outwardly from the contact face.

84. The tissue gripping device as claimed in claim 83, wherein the microgrooves are soft micro-pillars extending outwardly from the contact face such that the soft micro-pillars deform under sheering and normal force.

85. The tissue gripping device as claimed in any one of claims 82 to 84, wherein the surface of the contact face is treated with a hydrophilic, hydrophobic, knurled or abraded coating.

86. The tissue gripping device as claimed in any one of claims 70 to 85, wherein the head is rigid.

87. The tissue gripping device as claimed in any one of claims 70 to 86, wherein at least one lens, window or fiber optic cable is built into the head.

88. The tissue gripping device as claimed in any one of claims 70 to 87, wherein the head is constructed from transparent or translucent material.

89. The tissue gripping device as claimed in any one of claims 70 to 88, wherein the contact face comprises a plurality of openings of different sizes such that smaller openings are placed closer to the edge of the contact face.

90. The tissue gripping device as claimed in any one of claims 70 to 89, wherein the tissue gripping device is attachable to an endoscope, a drug administering device, an electrode or a medical retractor.

91. The tissue gripping device as claimed in any one of claims 70 to 90, wherein the head is attachable to a sensor.

92. The tissue gripping device as claimed in claim 91, wherein the sensor is capable of measuring at least one of the following: force, pressure, capacitance, temperature and pH.

93. The tissue gripping device as claimed in claim 92, wherein the sensor is an ultrasonic transducer, a magnetic resonance imaging coil, an optical sensor or lens, an optical coherence tomography sensor or a mechanical excitation and force transducer for elastography imaging.

94. The tissue gripping device as claimed in any one of claims 70 to 93, where the at least one opening is used to grasp and deliver a patch or a film into an orifice or a lumen.

95. The tissue gripping device as claimed in claim 94, wherein the patch or the film contains tissue, drugs, growth factor, cells or any combination thereof.

96. The tissue gripping device as claimed in any one of claims 70 to 95, wherein the contact face can seal an area of tissue.

97. The tissue gripping device as claimed in any one of claims 70 to 95, wherein the tissue gripping device comprises at least a first hollow member and a second hollow member and at least a first opening and a second opening in the contact face, such that the first opening is in flow communication with a first suction device through the first hollow member, and the second opening is in flow communication with a second suction device through the second hollow member wherein the first hollow member is not in flow communication with the second hollow member.

98. A tissue gripping device for use in association with a suction device, comprising:
a hollow member having a distal end and a proximal end;
a head, having a substantially convex contact face and an inlet, the inlet being attachable to the distal end of the hollow member;
the substantially convex contact face having at least one opening, the at least one opening being in flow communication with a suction device through the hollow member; and
a control device attachable to the proximal end of the hollow member.

99. A tissue gripping device for use in association with a clip and a suction device, comprising:
at least two members each having a distal end and a proximal end, wherein one of the at least two members is a flexible member and wherein one of the at least two members is a hollow member;
a head, having a contact face and an inlet, the inlet being attachable to the distal end of the hollow member;
a tool channel having a distal end and a proximal end;
the contact face having at least one opening, the at least one opening being in flow communication with a suction device through the hollow member;
a generally elongate tool housing formed in the head and configured to receive the clip wherein the tool housing is attachable to and in flow communication with the distal end of the tool channel;
a tool trench formed in the head configured to receive therein the distal end of the clip; and
a control device attachable to the proximal end of the hollow member and operably attachable to the proximal end of the tool channel, whereby the clip is ejected responsive to actuation by the control device from the tool channel and into the tool trench.

100. A tissue gripping device for use in association with a suction device and a bladder pump, comprising:
at least two members each having a distal end and a proximal end, wherein one of the at least two members is a flexible member and wherein one of the at least two members is a hollow member;
a head having a contact face and an inlet, wherein the inlet is attachable to the distal end of the hollow member;
an inflatable bladder, formed in the head, in flow communication with the bladder pump such that size dimensions of the head can be increased or decreased, responsive to the bladder pump;
the contact face having at least one opening wherein the at least one opening is in flow communication with the suction device through the hollow member; and
a control device attachable to the proximal end of the at least two members.

101. A surgical clip for use in association with a clip applying apparatus having a substantially elongate clip cartridge; the surgical clip being flexible, substantially bent or curved in stable equilibrium position and can be loaded into a substantially elongate clip cartridge such that the surgical clip's shape in loaded configuration is substantially more elongate than the surgical clip's shape in stable equilibrium position and wherein on being released from the elongate clip cartridge the surgical clip reverts back to its stable equilibrium position.

102. The surgical clip as claimed in claim 101, wherein the surgical clip is one of: rigid, elastic and super-elastic.

103. The surgical clip as claimed in claim 101 or 102, wherein the clip is composed of one of a shape memory alloy, a shape memory polymer, a biodegradable polymer, nitinol, stainless steel, titanium, and cross-linked polyurethane.

104. The surgical clip as claimed in any one of claims 101 to 103 wherein the clip has a sharp tip.

105. The surgical clip as claimed in any one of claims 101 to 104, wherein the cross section of the clip is one of: circular, rectangular or flat.

106. The surgical clip as claimed in any one of claims 101 to 105, wherein the surgical clip has a safety cap on the end.
